(19) United States
(12) Patent Application Publication
(10) Pub. No.: US 2009/0138979 A1
(43) Pub. Date: May 28, 2009

(54) MANIPULATING SP1 ACTIVITY TO IMPROVE THERAPEUTIC CLONING

(75) Inventors: Keith Latham, Havertown, PA (US); Rita Vassena, Barcelona (ES); Zhiming Han, Philadelphia, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Temple University - Of the Commonwealth System of Higher Education, Philadelphia, PA (US)

(21) Appl. No.: 12/323,041

(22) Filed: Nov. 25, 2008

Related U.S. Application Data
(60) Provisional application No. 60/990,656, filed on Nov. 28, 2007.

(51) Int. Cl. C12N 15/89; C12N 15/87

(52) U.S. Cl. 800/24; 800/21

(57) ABSTRACT

The observed over-expression of Sp1 target genes has inspired inventors to formulate a specific strategy for correcting many gene expression defects, and thus improve clone development. The invention is based on the belief that manipulating Sp1 activity can improve cloning. Inventors believe that cloning is inefficient in large part because of the continued expression of Sp1 target genes in the early cloned embryos, which causes clones to be very unlike normal embryos, and so makes them very unhealthy. Inventors propose that if over-expression of Sp1 target genes is prevented in early stage clones, this would greatly improve cloning efficiency by making the cloned embryos healthy again.

### STATEMENT REGARDING FEDERALLY SPONSORED RESEARCH OR DEVELOPMENT

This research was supported in part by U.S. Government funds (NIH Grant No. HD 43092), and the U.S. Government may therefore have certain rights in the invention.

### BACKGROUND OF THE INVENTION

1. Field of Invention

This invention relates to cloning by somatic cell nuclear transfer (SCNT).

2. Description of Related Art

Cloning by somatic cell nuclear transfer (SCNT) is a remarkable process that relies on the oocyte's ability to act upon the somatic nucleus and to transform it into a nucleus compatible with long-term embryonic development. This process of nuclear “reprogramming” is particularly remarkable considering the dramatic differences between somatic and early embryonic cells. These include fundamentally different cell cycles and cell cycle regulation (e.g., cleavage without growth), strikingly different gene expression profiles (Latham et al, 1991) revealed by two-dimensional gel electrophoresis, diverging modes of carbohydrate metabolism and energy production, a different array of amino acid transporters, glucose transporters, and ion transporters, (Van Winkle, 2001; Baltz et al, 1993; Baltz et al, 1991a; Baltz et al, 1991b; Pantaleon et al, 2001; Leppens-Luisier et al, 2001; Chi et al, 2000; Carayannopoulos et al, 2000; Hogan et al, 1991; Aghayan et al, 1992; Pantaleon and Kaye, 1998; Moley et al, 1998; Morita et al, 1994; Carayannopoulo et al, 2001), different mechanisms of osmoregulation and pH regulation (Baltz et al, 1993; Baltz et al, 1991a; Baltz et al, 1991b; Edwards et al, 1998; Zhao et al, 1996; Zhao et al, 2005), and dramatic differences in mitochondrial ultrastructure and activity (Shepard et al, 2000; Sathananthan and Trouson, 2000; Matsumoto et al, 1998; Shepard et al, 1998; Hillman and Tasca, 1969).

Over the course of the 50 years during which SCNT studies have been performed, first in amphibians (King and Briggs, 1955) and more recently in mammals (for review see Campbell et al, 2005, Latham K E, 2004 and references therein), it has become clear that the rate of success (i.e., development to term) is quite low (1-5%) (Rhind et al. 2003). Although incomplete nuclear reprogramming is often put forth as an explanation for this poor success, the nature of such a deficiency has never been defined.

The cell type-specific expression of transcription factors (both activators and repressors) likely results in a distinct global pattern of gene expression that provides a molecular signature that defines the differentiated state of a somatic cell. The expression of these transcription regulators, a priori, must be stable in order to maintain a stable state of differentiation, and indeed such seems to be the case (e.g., Hox genes in Drosophila). Thus, genes encoding transcription factors may be among the most difficult genes for the oocyte to reprogram during cloning. Failure to reprogram even a small number of key transcription factor genes could readily lead to a “ripple effect” resulting in aberrant expression of entire networks of downstream target genes.

All references cited herein are incorporated herein by reference in their entireties.

### BRIEF SUMMARY OF THE INVENTION

In one aspect, the invention relates to a method of increasing cloning efficiency of embryos, the method comprising manipulating Sp1 target genes expression to obtain a statistically significant increase in a fraction of embryos developing to birth.

In another aspect, the invention relates to a kit for increasing cloning efficiency of embryos, the kit comprising at least one of (a) a donor cell treated with siRNA to Sp1 mRNA or (b) an egg which is temporary neutralized by treating the egg with a sufficient amount of an Sp1 antibody or a dominant negative form of Sp1 such that aberrant overexpression of Sp1 target genes is reduced.

The transfer of nuclei from adult body cells (somatic cell nuclear transfer, SCNT) to eggs has been used successfully to produce cloned animals that are genetic copies of one another. This approach has tremendous potential in many areas, including preservation of endangered species, the propagation of genetically engineered animals that produce highly valuable biomolecules to be used for therapeutic purposes, the propagation of high-yield livestock, research into mechanisms of aging, and the production of stem cells that can be used to repair tissues or treat other diseases such as diabetes. These applications, however, have been hampered by the overall inefficiency of the cloning procedure, with only about 1 to 5% of SCNT constructs developing into live-born animals, depending on the species and donor cell types employed. Because of the costs involved in the current application of cloning technologies, any significant improvement in cloning success will have significant economic benefits in these areas.

Inventors have found that the reprogramming of gene expression in cloned embryos is very slow and incomplete (Chung et al., 2002; Gao and Latham, 2004; Gao et al., 2003), with clones aberrantly expressing >800 genes during early development. Inventors have found that >70% of these incorrectly expressed genes are targets of the Sp1 transcription factor. This most likely results from the rich supply of oocyte-derived Sp1 (Worrad et al., 1994) acting upon transcriptionally active genes in the donor nucleus.

The observed over-expression of Sp1 target genes has inspired inventors to formulate a specific strategy for correcting many gene expression defects, and thus improve clone development. The invention is based on the belief that manipulating Sp1 activity can improve cloning.

Cloning is inefficient in large part because of the continued expression of Sp1 target genes in the early cloned embryos, which causes clones to be very unlike normal embryos, and so makes them very unhealthy. Inventors propose that if this “accidental” over-expression of Sp1 target genes is prevented in early stage clones, this would greatly improve cloning efficiency by making the cloned embryos healthy again.

### BRIEF DESCRIPTION OF SEVERAL VIEWS OF THE DRAWINGS

FIG. 1. Real time PCR derived expression patterns of selected genes at the one-cell (Panel A) and two-cell (Panel B) stage cultured in different media with or without α-amanitin. Y axes indicate the relative fold change to fertilized embryos cultured in KSOM (reference treatment, expression adjusted to =1.0). F=fertilized embryos; C═SCNT embryos, P=parthenotes; A=amanitin treatment, K=KSOM culture medium, M=MEMα culture medium. Significant difference among kind of embryos and culture media are indicated as follows: a: p<0.1; b: p<0.05; c: p<0.01; d: p<0.001.

FIG. 2. Number of genes differentially expressed in SCNT, in vivo fertilized and parthenogenetic embryos at the late one-cell stage. Those genes that displayed α-amanitin dependent reductions in mRNA abundance were judged to be transcribed, while those that did not were judged to be non-transcribed.

FIG. 3. Number of genes differentially expressed in SCNT, in vivo fertilized and parthenogenetic embryos at the two-cell stage. Those genes that displayed α-amanitin dependent reductions in mRNA abundance were judged to be transcribed, while those that did not were judged to be non-transcribed.

FIG. 4. GO functional annotation of α-amanitin sensitive transcripts upregulated in SCNT compared to fertilized and parthenogenetic embryos at the two-cell stage. Numbers beside each category indicate the number of mRNA in that category.

FIG. 5. Comparison between expected fold change based on microarray analysis (black bars) and observed fold change by real time PCR (white bars) for selected genes in SCNT embryos at the one-cell (Panel A) and two-cell (Panel B) stage.

FIG. 6. Ingenuity Pathway Analysis output example of an interaction network between transcription factors (light blue) and other upregulated genes in SCNT two-cell embryos.

### DETAILED DESCRIPTION OF PREFERRED EMBODIMENTS OF THE INVENTION

The inefficiency of the currently used SCNT procedure creates severe practical limitations for therapeutic cloning, by creating excessive need for human oocytes to serve as SCNT recipients and by creating other societal concerns related to access to the technology. Other technical concerns for therapeutic cloning relate to the safety of the approach for cell-based therapies to treat illnesses. For example, concerns exist related to loss of epigenetic information (see e.g., Mann et al., 2003; Ohgane et al., 2001; Humpherys et al., 2001), which may be a specific consequence deficient reprogramming in clones leading to poor adaptation to standard embryo culture environments (see Gao and Latham, 2004; Gao et al., 2003, 2004a; Latham, 2005).

To resolve these problems, it is necessary to improve the efficiency of the cloning procedure, and to alleviate safety concerns by ensuring correct nuclear function. Inventors have found that cloned embryos suffer from an “identity crisis.” They express some genes that are expected to be expressed in normal embryos, but they also continue to express many genes that were expressed in the adult cell, but which should not be expressed in embryos (e.g., Gao et al., 2003; Latham 2005). This means that clones do not tolerate embryo culture systems as do normal embryos. As a result, the cells of cloned embryos rapidly become unhealthy and most become arrested developmentally. Additionally, exposure of clones to sub-optimal culture likely creates many of the epigenetic abnormalities that have been reported. To make cloning work better, inventors propose to make clones act more like normal embryos; this means finding a new way to make clones express the correct array of genes.

The invention is based upon a discovery that cloning is inefficient in large part because of the continued expression of Sp1 target genes in the early cloned embryos, which causes clones to be very unlike normal embryos, and so makes them very unhealthy. Inventors propose that if this “accidental” over-expression of Sp1 target genes is prevented in early stage clones, this would greatly improve cloning efficiency by making the cloned embryos healthy again.

The term “Sp1 target genes” as used herein means genes that are targets of the Sp1 transcription factor and possess an Sp1 binding site (5′-(G/T)GGGCGG(G/A)(G/A)(C/T)-3′, where (G/T) indicates that the domain will bind a guanine or thymine at this position).

The term “improve cloning efficiency” as used herein means obtaining a statistically significant increase of cloning efficiency relative to that achieved by prior developments when only about 1 to 5% (2% for mice) of SCNT constructs developing into live-born animals. Preferably, cloning efficiency can be improved to at least 10% when Sp1 activity is being manipulated.

To investigate ways of increasing cloning efficiency, inventors propose conducting two lines of experiments. One approach is designed to turn off Sp1 target genes in the donor cell nucleus before nuclear transfer. In that approach, Sp1 protein expression in donor cells prior to nuclear transfer, in order to turn off Sp1 target genes, will be reduced. Reduction of Sp1 protein expression in donor cells is considered successful when reduced by at least 90% (Yin et al., 2006).

The second approach is to block Sp1 target gene expression after nuclear transfer. In that approach, the Sp1 protein present in the egg is temporary neutralized by injecting a Sp1 antibody or a dominant negative form of Sp1. In the second approach, Sp1 activity in the cloned embryo is reduced sufficiently to reduce the aberrant overexpression of these 880 target genes whilst permitting expression of other essential Sp1 target genes.

Additionally, because the protein complex CRSP is required for Sp1 activation (Ryu et al. 1999), an alternative approach will be to target CRSP subunit expression in donor cells and cloned constructs.

Components or subunits of the “Cofactor Required for Sp1” (CRSP) complex as identified previously (Ryu and Tjian, 1999), with transcription activity associated with polypeptides of 33K (Crsp9, NM_{—}025426), 34 K (Crsp8, NM_{—}026896), 70 K (Crsp7, NM_{—}027485), 77 K (Crsp6), 85 K, 100 K, 130 K(Crsp3, NM_{—}027347), 150 K(Crsp2, NM_{—}001048208), and 200 K (Pparbp, NM_{—}013634), and particularly CRSP150 (Crsp2, NM_{—}001048208), CRSP130 (Crsp3, NM_{—}027347), and CRSP33 (Crsp9, NM_{—}025426) proteins, which were found to be associated with peak activity. If CRSP subunits are targeted, enhanced specificity of treatment toward Sp1 target genes should be achieved by specifically targeting components of the CRSP/Med2 complex (Taatjes and Tjian, 2004). Ryu S., Tjian R. Purification of transcription cofactor complex CRSP.Proc Natl Acad Sci USA. 1999 96(13):7137-42. Taatjes D J, Tjian R. Structure and function of CRSP/Med2; a promoter-selective transcriptional coactivator complex. Mol. Cell. 2004 14(5):675-83.

After Sp1 or CRSP subunit protein manipulation, cloned embryos will be evaluated for improvement in gene expression pattern and developmental potential. This will be achieved by microarray-based comparisons of gene expression at the 2-cell stage between treated and untreated clones and controls, as well as comparison of blastocyst development rate, blastocyst cell number, allocation of cells to the inner cell mass, and developmental potential to term, as described in the attached manuscripts and other publications from inventors laboratory (Chung et al., 2002; Gao and Latham, 2004; Gao et al., 2003, 2004a).

If either of the two proposed approaches is successful, cloning efficiency would increase dramatically. This would have substantial economic and scientific benefits, and unlock the enormous potential of this technology for therapeutic cloning. Inventors expect that, given the number of Sp1 target genes being overexpressed in clones, these approaches will be highly effective at making the clones act more like normal embryos. This will be reflected in an increased rate of development in culture using media optimized for normal mouse embryos, and in a gene expression pattern more like that of normal embryos, as revealed by microarray analysis. The clones will thus develop better through preimplantation development and will avoid acquiring many of the epigenetic derangements in gene expression that have been observed, thereby enhancing viability and health of cloned progeny.

Inventors believe the approach of targeting a specific transcriptional target gene set has many advantages over non-specific approaches that were undertaken by other laboratories. These other approaches often involve exposing clones to or donor cells to metabolic inhibitors (e.g., DNA methylation inhibitors, or inhibitors of DNA acetylation/deacetylation) in the hope that this will somehow make clones healthier. The over-riding deficiency of such approaches is that these inhibitors have broad, highly non-specific effects on gene expression, and may actually increase relative risks for therapeutic applications by causing gene regulation to be disregulated in unpredicted ways. The approach offered by the current invention provides a way to achieve highly specific effects and is focused on a specific array of target genes, without any permanent, heritable genetic or epigenetic manipulation. This approach should yield much more predictable and consistent results more highly suited to the needs of therapeutic cloning.

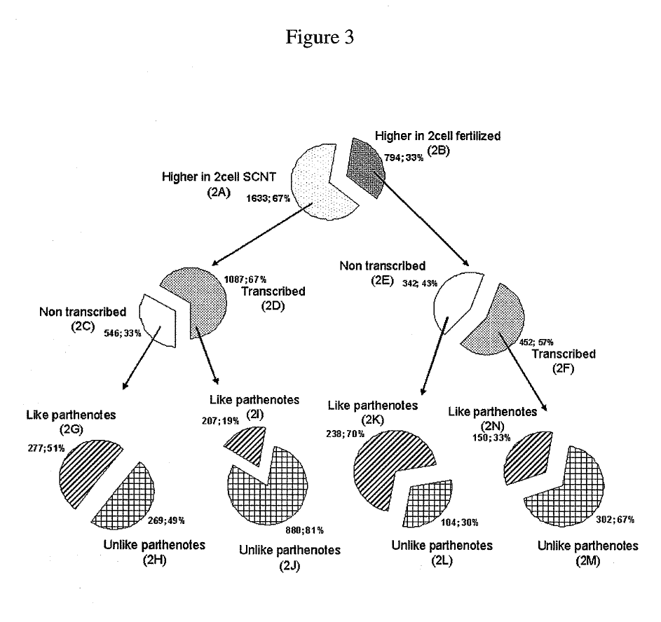

Inventors undertook a detailed microarray gene expression study, comparing cloned embryos with parthenogenetic and fertilized control embryos at the 1- and 2-cell stages (FIG. 3). Clones were first compared to fertilized embryos; a set of genes that are over-expressed in clones and also dependent on transcription (α-amanitin sensitive; FIG. 3 set 2D) was identified. It was determined which of these genes were also different between clones and parthenotes (FIG. 3 set 2J). Parthenotes provide valuable controls because they are activated from the same pools of ooctyes and develop synchronously alongside clones, thereby controlling for differences related to activation and absence of a fertilizing sperm, and eliminating effects related to simple asynchrony. This final filter on the array data yielded a set of genes (n=880; FIG. 3 set 2J), which are over-expressed in cloned 2-cell embryos as compared to control embryos. The over-expression of these 880 genes is, therefore, a feature of the cloned embryo phenotype, which serves as an indicator of disruption in gene regulation in the cloned embryos.

Examining the promoter regions for these over-expressed genes, inventors found that most (73%) of these genes are activated in response to Specificity Factor 1, or Sp1 (see Table 1 below). This representation of Sp1 target genes is statistically highly significant for both mouse and the human orthologs (p<10⁻⁶ to 10⁻¹¹). While a small number of other transcription factor binding sites were statistically over-represented, none of these encompassed such a large fraction of affected genes as Sp1.

TABLE 1
Results of PRIMA analysis of promoters of genes up-regulated in clones

M00255 [GC_box] (length 14): 1.2e−11 (1), Ortholog = 3.5e−09 (3)
M00196 [Sp1] (length 13): 2.8e−11 (2), Ortholog = 7.5e−10 (1)
M00932 [Sp-1] (length 13): 4.2e−10 (3), Ortholog = 4.5e−09 (4)
M00931 [Sp-1] (length 10): 6.8e−10 (4), Ortholog = 2.4e−09 (2)
M00933 [Sp-1] (length 10): 3.4e−06 (5), Ortholog = 1.8e−06 (5)
M00008 [Sp1] (length 10): 5.3e−05 (6), Ortholog = 2.8e−06 (6)
M00803 [E2F] (length 6): 7.1e−04 (14), Ortholog = 3.8e−05 (9)
M00982 [KROX] (length 14): 1.5e−04 (8), Ortholog = 1.6e−03 (16)
M00025 [Elk-1] (length 14): 9.2e−04 (16), Ortholog = 7.3e−04 (11)
M00108 [NRF-2] (length 10): 2.7e−03 (24), Ortholog = 2.3e−05 (7)
M00653 [OCSBF-1] (length 5): 2.5e−04 (12), Ortholog = 7.4e−03 (30)
M00113 [CREB] (length 12): 2.9e−03 (26), Ortholog = 2.9e−03 (20)
M00440 [CG1] (length 11): 3.0e−03 (27), Ortholog = 4.0e−03 (24)

Experimental Design and Methods:

The two approaches for reducing expression of the 880 Sp1 target genes in cloned embryos, and thereby affect cloned embryo development will now be described in detail.

The first approach is to reduce Sp1 target gene expression in donor cell nuclei, so that these genes will be less likely to be over-expressed in the cloned embryo.

By reducing the expression of this protein before nuclear transfer, the Sp1 target genes will be down-regulated. These genes, once down-regulated, should remain down-regulated after nuclear transfer, resulting in clones with greater viability and characteristics more like normal embryos.

General Description of Methods: Cultured donor cells are treated with siRNA; to the Sp1 mRNA (e.g., 5′-AAAGCGCUUCAUGAGAGGUGA-3, Pore et al., 2004) to suppress the expression of Sp1. This treatment can be accomplished by standard methods known in the art, including, for example, electroporation and nucleofection of the siRNAs into the cells.

Nucleofection has been adapted for siRNA studies, and has been highly effective in cell types for which transfection is otherwise difficult (Yin et al., 2006; Hagemann et al., 2006). This method is preferred, as it is essential not to genetically modify permanently the donor cell genome. The nucleofection can be performed 12-24 h prior to nuclear transfer. Sp1 knockdown is evaluated by Western blotting and/or immunofluorescence; the treatment duration is adjusted to reduce Sp1 activity and target gene expression without compromising cell viability. The optimized treatment is applied for cloning studies.

The Sp1-manipulated cells are employed for cloning. Non-manipulated cells are employed as comparison controls to confirm efficacy. The nuclei are isolated from these cells and introduced into mouse eggs by piezo-assisted miucroinjection or electrofusion (the spindle and chromosomes are removed before nuclear transfer) as described (Gao et al., 2001; Gao and Latham, 2004; Gao et al., 2003, 2004a). The eggs are activated and allowed to develop as in our earlier studies (see Gao et al., 2003, 2004a) and as described below.

Markers of Efficacy:

(1) Microarray analysis is conducted to confirm effect on 880 target genes (FIG. 3 set 2J). Two cell stage clones and controls are examined by microarray analysis as described below. Success will be indicated by the restoration of levels of expression of a substantial fraction of these genes to a normal range resembling normal fertilized embryos.

(2) Blastocyst formation and quality. Effective treatment will result in a significantly increased percentage of embryos achieving blastocyst stage, as well as significantly increased quality of blastocysts as measured by total cell number and relative allocation between inner cell mass and trophectoderm lineage, with statistical significance revealed through standard statistical tests. Clones are fixed and examined at the blastocyst stage to determine total cell number and number of cells allocated to the inner cell mass.

(3) Term Development: Manipulation of Sp1 activity should yield a statistically significant increase in the fraction of embryos developing to birth. Embryos are transferred to pseudopregnant foster mothers using standard methods for development to birth (Hogan et al., 1994).

Anticipated Results: Inventors anticipate that silencing of the Sp1 target genes in the donor cells, preferably 12-14 h prior to nuclear transfer will permit sufficient time for chromatin remodeling, a process by which the Sp1 target genes should become silenced. Once this has occurred, inventors expect that these genes will cease to be precociously activated by the Sp1 present in the egg after nuclear transfer. This will make the clones more like normal embryos, which will increase their developmental potentials.

Aside from the immediate benefit of improving cloned embryo development, inventors anticipate that these studies will establish a new paradigm for studying how genes and the other egg components interact. This will allow us to pursue many additional studies to unlock the secrets of how each life begins.

The second approach is to reduce Sp1 activity in the cloned embryo sufficiently to reduce the aberrant overexpression of these 880 target genes whilst permitting expression of other essential Sp1 target genes. The second approach involves the selective reduction in Sp1 protein function in the cloned embryos after nuclear transfer. By reducing the Sp1 activity after nuclear transfer, the 880 (FIG. 3 set 2J) aberrantly over-expressed Sp1 target genes will be down-regulated. These clones will be much more like normal embryos in their characteristics and developmental potential. The main requirement of this approach is that Sp1 function only be reduced temporarily, because Sp1 expression will be required for embryo survival at later stages. By suppressing Sp1 genes only during the first 1-2 cell cycles in clones (a period of about 2 days at the start of the clone embryo life), an opportunity will be provided for the Sp1 target genes to be inactivated, thus facilitating reprogramming so that clones will initially act more like normal embryos. Subsequently, as Sp1 activity is restored via embryonic Sp1 expression, which increases during development (Wang and Latham 2000), embryo development should proceed, and clones should have enhanced ability to develop to birth.

General Description of Methods: Eggs are isolated after ovulation as in our earlier studies (see Gao et al., 2003, 2004a), and as described below, and microinjected either with an antibody to Sp1, or with mRNA that will direct the eggs to express a dominant negative form of Sp1 (Al-Sarraj et al., 2005). For both antibody and dominant negative mRNA injections, a series of doses is employed in order to define the optimum range of effect. Controls include non-injected eggs and eggs injected with water only. After microinjection, the eggs are employed for cloning using cumulus cell nuclei as in our previous studies (see Gao et al., 2003, 2004b, 2005) and as described below.

Markers of Efficacy:

(1) Microarray analysis to confirm effect on 880 target genes (FIG. 3 set 2J). Two cell stage clones and controls are examined by microarray analysis as described below.

(2) Blastocyst Formation and Quality: Effective treatment will result in an increased percentage of embryos achieving blastocyst stage, as well as increased quality of blastocysts as measured by total cell number and relative allocation between inner cell mass and trophectoderm lineage. Clones are fixed and examined at the blastocyst stage to determine total cell number and number of cells allocated to the inner cell mass.

(3) Term Development: Manipulation of Sp1 activity should yield statistically significant increase in the fraction of embryos developing to birth. Embryos are transferred to pseudopregnant foster mothers using standard methods for development to birth.

Anticipated Results: Inventors anticipate that a well-controlled, temporary suppression of Sp1 content in the egg will prevent the aberrant expression of most, if not all of the 880 genes identified by microarray analysis to be overexpressed in untreated clones. Inventors also expect that clones will display phenotypes more like normal embryos, and will have increased potential to develop to term. It will be important to achieve an optimum reduction in Sp1 activity, one that alleviates over-expression of Sp1 target genes in the newly constructed clones, but which does not prevent the essential Sp1 target gene expression as development proceeds. The combination of developmental viability checks and microarray analyses will reveal the degree to which this optimum is achieved and pinpoint the appropriate dosage for the two approaches of siRNA knockdown in donor cells or dominant negative treatment after nuclear transfer.

In addition to targeting Sp1 directly, the same two approaches can be taken to target components of the CRSP complex, which is required for Sp1 mediated gene transcription.

To determine the degree to which inefficient reprogramming of transcription factor genes may underlie poor cloning success, to examine clones for disruption in the expression of other genes, and to identify specific biological processes that are likely disrupted as a consequence, inventors analyzed the transcriptome of clones immediately following SCNT using microarrays. In contrast to previous studies that focused on surviving clones of advanced development (Humpherys et al, 2002; Smith et al, 2005), inventors focused on the first two cell cycles, because these stages encompass the earliest interactions between ooplasm and donor nuclei, and because aberrant gene regulation at these early stages can have profound consequences for long-term development. The goal was therefore to determine to what degree SCNT embryos at these early stages resemble normal embryos of high developmental potential, and to what degree the somatic cell program might remain expressed.

Inventors found that, although the transcript profiles of SCNT and fertilized embryos are quite similar at the one-cell stage, aberrant gene transcription is nevertheless evident even at this early stage, along with apparent disruptions in the regulation of maternally encoded (i.e., oocyte-accumulated) mRNAs. During the two-cell stage, as transcriptional activation ensues, the number of aberrantly transcribed genes in SCNT embryos increases by nearly two orders of magnitude to nearly 1,000 genes, indicating a substantial continued expression of the somatic cell program. As predicted, the aberrantly expressed mRNAs include many involved in transcription, and also many involved in mRNA processing, oxidative phosphorylation, metabolism, protein biosynthesis, protein degradation, protein modification, and transmembrane solute transport.

Materials and Methods:

Preparation and Collection of Mouse Embryos:

Ovulated eggs were obtained from adult (B6D2)F₁ females 8-12 weeks of age by superovulation as described (Chung et al., 2002; Gao et al., 2003, 2004a). Adherent cumulus cells were removed by hyaluronidase treatment and the eggs were cultured in CZB medium supplemented with glucose (Chung et al., 2002). SCNT was performed as described (Chung et al., 2002; Gao et al., 2003, 2004a). At the end of the procedure, cloned constructs were activated by 5.5 h of culture in Ca²⁺-free CZB medium supplemented with 10 mM Sr²⁺ and 5 μg/ml cytochalasin B (Chung et al., 2002). Cloned constructs were cultured in minimal essential medium alpha formulation (MEMα) medium as described (Gao et al., 2004a) with or without α-amanitin (24 μg/ml). For SCNT, adherent adult cumulus cells (presumably G₁ phase) from ovulated oocytes were employed as nuclear donors. Diploid parthenogenetically activated embryos were obtained using the same activation protocol of clones. The parthenotes were obtained from the same pools of oocytes used to make cloned embryos and were activated at the same time. Parthenogenetic embryos resemble normal fertilized embryos with respect to culture requirements, but have the added advantage that they are activated and develop in close temporal synchrony with the activated cloned embryos. Embryos fertilized in vivo (henceforth referred to as fertilized) were obtained by mating (B6D2)F₁ mice after injection of females 8-12 weeks of age with Pregnant Mare Serum Gonadotropin (PMSG) and human Chorionic Gonadotrophin (hCG), as described (Chung et al., 2002; Gao et al., 2003, 2004a). Cloned, parthenogenetic, and fertilized embryos were cultured at 37° C. in an atmosphere of 5% CO₂ in air.

RNA Extraction, Labeling, and Hybridization:

For each experimental/treatment group, four pools of 20 embryos were collected and transferred to 20 μl of extraction buffer (Picopure, Arcturus). The tube was incubated at 42° C. for 30 min and then stored at −70° C. RNA extraction was performed with the Picopure RNA extraction kit according to manufacturer instructions for small sample preparation. For each sample, the mRNA population was reverse transcribed. The cDNA was employed for a first round of in vitro transcription, followed by random priming and a second round of reverse transcription and in vitro transcription to achieve a linear amplification (Affymetrix Small Sample Technical Bulletin, www.affymetrix.com) with the following minor modifications: the initial volume for mRNA annealing was raised to 5 μl, and the conditions for reverse transcription were 30 min at 42° C. followed by 30 min at 45° C. to increase the reaction efficiency in GC rich regions of mRNA. The final yield of biotinylated cRNA was 28.5 to 83.4 μg for one-cell stage embryos and 26 to 88.5 μg for two-cell stage embryos; 20 μg of cRNA per replicate were fragmented and 10 μg hybridized to Affymetrix MOE 430 2.0 Gene Chips in the Penn Microarray Facility, then washed and stained on fluidic stations, and scanned according to the manufacturer's instructions.

Microarray Data Analysis:

Microarray Analysis Suite 5.0 (MAS, Affymetrix) was used to quantify microarray signals with default analysis parameters and global scaling to target a mean equal to 150 signal units. Quality control parameters for all samples were within ranges shown in Table 2. Tabular data for all samples are available at the Gene Expression Omnibus (GEO) repository (www.ncbi.nlm.gih.gov/geo). The MAS metric output was loaded into GeneSpring v7 (Silicon genetics) with per chip normalization to the 50^{th} percentile and per gene normalization to the median. To minimize false positive signals, only genes called “Present” in at least three out of four replicates in one embryo kind/condition were used for further analysis with all statistical packages. The K-means hierarchical clustering (HCL) of GeneSpring v7 was used among samples at the same developmental stage to divide them into groups based on their expression patterns and to produce groups with a high degree of similarity within groups and low degree of similarity between groups.

It is important to note that, although the Affymetrix MOE430 2.0 array interrogates one gene with every probe set, 14.7% of the genes present on the array are represented by more than one probe set. All analyses described were performed using the Affymetrix probe set lists, except when noted where gene numbers were used to avoid redundancy.

The filtered MAS metrics output was loaded into TIGR-MEV v3.0.3 (Saeed et al, 2003). The Statistical Analysis of Microarray (SAM; Tusher et al., 2001) algorithm was applied to identify genes with significant differences among samples at the 1% false discovery rate (FDR).

Fold-changes of expression differences between stages and conditions were calculated following SAM analysis. The resulting lists of differentially expressed (≧two-fold) genes were imported into Expression Analysis Systematic Explorer (EASE, version 2.0) to analyze gene ontology for over-representation (Hosack et al., 2003). EASE is an algorithm designed to analyze a list of candidate genes against a set population (in our case the list of genes detected on the GeneChip) and to report a score that is the expression of the likelihood of over-representation in the Gene Ontology (GO) annotation categories for biological process, molecular function, or cellular component. The EASE score was calculated for likelihood of over-representation of annotation classes, and only GO biological processes with an EASE score less than 5% are shown. It is important to note that a significant EASE score does not relate to an increased fold-change or overall expression significance, but merely a higher than expected number of transcripts falling into a GO annotation category.

The filtered list of transcripts over-expressed in clones versus fertilized and parthenogenetic embryos, and also with α-amanitin sensitive (i.e., reduced by α-amanitin treatment) expression, and different in expression from parthenogenetic embryos at the two-cell stage was further imported into Ingenuity Pathway Analysis (IPA, www.ingenuity.com) in order to detect networks detailing physical association or functional interaction among transcripts falling into different GEO annotation categories.

Quantitative RT-PCR Analysis:

Groups of 25-50 embryos were collected, and total RNA was isolated as described above. Thirteen genes were selected for analysis at the one-cell and two-cell stage, and their mRNAs quantified by reverse transcription followed by real time PCR (qRT-PCR). The corresponding ABI TaqMan gene expression IDs were: Zar1 (Mm-00558078), Yy1 (Mm-00456392_ml), Fos (Mm00487425_m1), Cpa1 (Mm_{—}00465942_m1), H1foo (Mm00506768_m1), Zfp352 (Mm-02528443_s1), Por (Mm00435876_m1), Eif3s12 (Mm-00503812_ml), Maf1 (Mm-00593524_g1), Klf4 (Mm-00516104_m1), Sra1 (Mm-00491755_m1), Uqcrb (Mm-00835346_gH), Psmc3 (Mm-00477177_m1). Three replicates were used for each qRT-PCR reaction, and each mRNA was analyzed 2-3 times per replicate; minus RT and minus primers/probe reactions served as controls. Quantification was normalized to the endogenous histone H2A [Mm-00501974_s1, (Hisst2ah2aa10) within the log linear phase of the amplification curve using the comparative Ct method (ABI PRISM 7700 Sequence detection System, user bulletin 32). These mRNAs were selected to be examined by qRT-PCR because of their apparent abundances as judged by the microarray hybridization signals and as representatives of specific functional categories (see Results).

Results:

Experimental Design:

The objectives of this study were to determine the timing and extent of nuclear reprogramming during the first two cell cycles of SCNT embryo development, and to identify specific genes or categories of genes that could account for the observed differences in phenotype between SCNT and fertilized embryos. To meet these objectives, inventors adopted a microarray-based approach for transcript profiling that has been used successfully for mouse oocytes and preimplantation embryos (Zeng et al, 2004, 2005; Pan et al, 2005).

Although simple in concept, such studies are complicated by technical aspects of SCNT embryo production and culture. First, it is difficult to obtain in vivo fertilized embryos that are developing in close synchrony with SCNT embryos, so that effects of asynchrony on relative mRNA abundances could arise. To control for possible effects of asynchrony, we employed parthenogenetic controls, which are activated at the same time as SCNT embryos using the same method, and from the same pools of eggs as those employed to prepare the SCNT embryos. The use of parthenogenetic controls also accounts for possible differences that might be related to absence of a fertilizing sperm and activation in response to chemical treatment rather than sperm factors. For this reason, parthenogenetic controls provided a significant advantage over, for example, in vitro fertilized embryos, as a control for possible asynchrony, because they addressed additional aspects of the procedures used to produce SCNT embryos.

Second, SCNT embryos display radically altered culture medium preferences when compared to normal embryos (Chung et al, 2002). No single culture medium has yet been identified that is optimized for both SCNT and normal embryos. In fact many SCNT embryos arrest in media optimized for embryo culture, and many fertilized embryos arrest in the somatic cell culture media favored by SCNT embryos (Chung et al, 2002). Because our objective was to explore the limits and timing of reprogramming, it was essential that the analyses be performed on embryos of the highest developmental potential and cultured in the best media available for each type of embryo. This would avoid comparisons between embryos that are developmentally viable and embryos that are already developmentally arrested, or between two kinds of embryos both of which are known a priori to be compromised. Such comparisons would yield artifactual results that would be unrelated to basic questions related to nuclear reprogramming and how well clones resemble normal embryos.

Inventors adopted the strategy of employing the best available culture media for each kind of embryo, namely MEMα for SCNT embryos and KSOM for parthenotes and fertilized embryos. SCNT embryos develop very poorly in KSOM even to the four-cell stage, making an analysis of SCNT embryo in this medium uninformative (Chung et al, 2002). Fertilized embryos and parthenogenetic embryos have been cultured in MEMα. Although this medium has been found to be superior to a number of grossly sub-optimum media, KSOM remains superior to MEMα for such embryos (Chung et al, 2002). Inventors were able to compare embryos of all three classes under those culture conditions that support the highest in vitro efficiency achievable beyond the first two cell cycles and, more importantly, to display the greatest rates of development to the blastocyst stage, the highest quality of blastocysts, and the most consistent rates of development to term achievable. This permitted the microarray analysis to reveal specific effects of SCNT and nuclear function without concern that such differences were being contributed by less specific deficiencies related to simple developmental arrest.

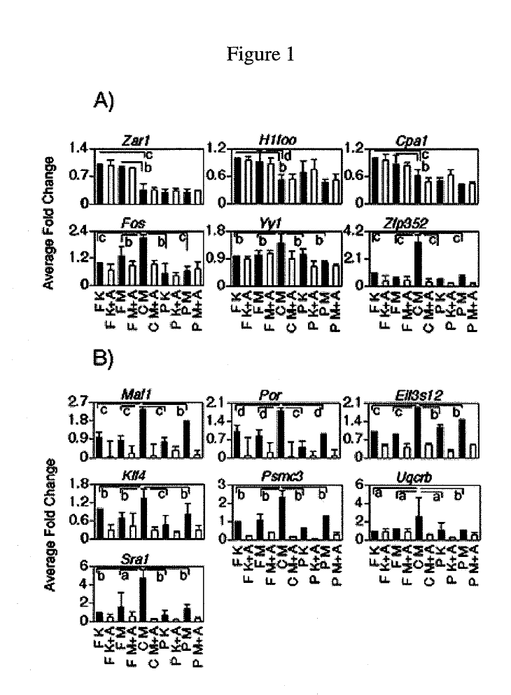

This strategy, however, creates a secondary need to account for possible effect of the different culture media. To resolve this issue, inventors applied two sets of controls. In one control study, inventors undertook an independent microarray comparison between fertilized two-cell embryos cultured in either KSOM or MEMα, using the same developmental time point and data analysis parameters described above. This comparison between fertilized embryos cultured in the two media yielded a set of 145 genes, the expression of which could potentially be altered by the choice of culture medium. This set of media-sensitive genes was later compared to the lists of genes differentially expressed between two-cell stage SCNT and normal embryos in order to reveal potential effects of culture medium. Inventors observed only 12 genes in common between the media-sensitive list and the lists of genes altered in two-cell SCNT embryos indicating that the potential effect of the culture systems on the overall microarray results is highly limited. As a second test for possible effects of culture medium, we employed qRT-PCR analysis to compare gene expression between SCNT, fertilized, and parthenogenetic control embryos cultured either in KSOM or MEMα (FIG. 1). These analyses revealed little if any variation between samples of fertilized control embryos cultured in different media (compare FK and FM in FIG. 1). Although for some of the genes assayed slightly greater differences were observed between parthenotes cultured in the two media, qualitatively identical directional differences in gene expression were seen even between SCNT and parthenotes, regardless of the media employed. Collectively, these data indicate that the culture media employed for maintaining the highest developmental potential amongst SCNT and control embryos while in culture did not adversely affect the discovery of differences in gene expression. This result confirms the robustness of the statistical analysis.

The final requirement for the array analysis was to be able to distinguish between effects on maternal transcript populations and effects on transcribed genes. To address this requirement, inventors included in the experimental design for both microarray and qRT-PCR experiments SCNT and fertilized embryos that were cultured in the presence of α-amanitin, a potent RNA polymerase II inhibitor. The treated embryos would thus display α-amanitin-dependent reductions in mRNA abundance for transcribed genes.

The approach to identify sets of differentially expressed genes used herein incorporated stringent parameters for false discovery rate, statistical significance of difference, and fold cutoff, combined with sequential filtering of gene sets based on differential expression first between SCNT and fertilized embryos, then between SCNT and parthenogenetic controls, and finally distinctions based on α-amanitin sensitivity. The gene sets obtained are highly reliable, and thus capable of providing significant new insight into how genes are differentially regulated between SCNT and control embryos, and hence the extent and timing of nuclear reprogramming.

Overview of Microarray Results:

The microarray data sets obtained in this study are available in tabular form from the Gene Expression Omnibus Repository (www.ncbi.nlm.nih.gov/geo). Among the entire series of samples, expression of between 13,230 and 18,500 mRNAs was detected (Table 2). This range reflects differences in the complexity of the mRNA populations of different stages/treatments of embryos. The quality control parameter for all the samples were within the following ranges: scale factor 0.6 to 1.9 (accepted range: 0.5 to 5.0), and background 35.8 to 64.5 (accepted range: 20 to 100); percent IDs detected 29.4 to 41.1; actin 3′/5′ signal ratio 3.3 to 12.4; GADPH 3′/5′ signal ratio 1.5 to 7.7 (Table 2). The quality control data are in agreement with that reported in two other studies using the same array platform (Zeng et al, 2004; Pan et al, 2005) as well as within the ranges recommended by Affymetrix. All the quality control parameters, as well as the internal and spiked controls in place to ensure correct mRNA processing and preparation, confirmed that the datasets obtained were of high quality.

It is often assumed that reprogramming must occur within hours of nuclear transfer. Published studies, however, indicate that clones manifest unusual characteristics during these early stages indicative of slow or incomplete reprogramming (Gao et al., 2003, 2004a; review, Latham, 2004, 2005). No study to date has attempted to measure the degree of similarity or difference between SCNT and fertilized embryos. Inventors used K-means hierarchical clustering (HCL) to ascertain the overall similarities/differences of embryos derived from the different treatments. At both developmental stages, replicate samples of the same kind/condition clustered together and apart from other embryo kinds/conditions, which indicates that SCNT are indeed significantly different from control embryos with respect to transcriptome composition. Additionally, this clustering pattern indicates a high degree of reproducibility and small biological variability among samples of a given kind of embryo. It is noteworthy that the HCL output of one-cell stage embryos grouped embryos by kind and treatment, indicating that SCNT embryos at this stage of development are already different from both normal and parthenogenetic embryos. Moreover, the clustering of the α-amanitin treated samples apart from non-treated ones indicates that the α-amanitin effect is already sizeable at this early stage.

Three other aspects of the data argue for an early effect of the donor nucleus on the SCNT embryo phenotype. First, it was observed that the two-cell stage samples treated with α-amanitin (both fertilized and SCNT embryos) are distinct from the three non α-amanitin-treated groups, but that the α-amanitin treated samples retain their cluster grouping by kind of embryo (i.e., SCNT embryos remain separate from fertilized embryos). This indicates that the maternal (i.e., not diminished by α-amanitin treatment) mRNA population is regulated differently between SCNT and fertilized embryos due to the difference in nuclear origin, a point that will be addressed further below. Second, one-cell parthenogenetic embryos cluster apart from both SCNT and fertilized embryos, at a position intermediate between the latter two groups. This indicates that even before the first cleavage division, the cloned embryo transcriptome has diverged even from that of parthenogenetic controls, which are activated simultaneously from the same pool of eggs and developing in close synchrony with SCNT embryos. Third, it was observed that the degree of difference between SCNT and fertilized embryos increases between the one-cell and two-cell stages. If nuclear reprogramming occurred rapidly after SCNT, then we would not expect a large increase in the degree of difference between SCNT, parthenogenetic, and fertilized embryos as development proceeds. The two-cell HCL plot instead reveals an increasing divergence between the three classes of embryos, indicating that the donor cell nuclei exert a strong effect on phenotype as the embryo proceeds through embryonic genome activation.

Global Changes in mRNA Population During the First Embryonic Cell Cycle:

Inventors are trying to ascertain how well the donor cell genome is silenced after transfer into recipient eggs. Two scenarios could be envisioned. In the first one, as the one-cell embryo acquires the capacity to undertake gene transcription (Latham et al, 1992), an array of donor cell genes could be transcribed before the first cell division. Indeed, the overall rate of transcription in clones might be increased due to the original chromatin state of the donor genome. Alternatively, because the ooplasm establishes a transcriptionally repressive state within the early embryo a (Latham et al, 1992), the donor cell genome may become highly transcriptionally repressed. The current microarray data distinguish between these alternatives, and also provide an opportunity for identifying aberrantly expressed genes. Moreover, they provide new information about the fate of maternal transcripts in clones.

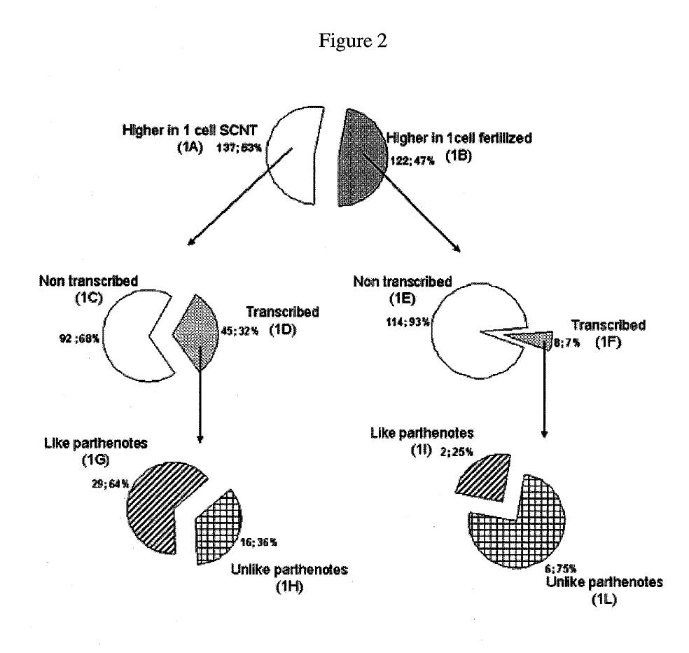

Inventors found 259 mRNAs that were differentially expressed between SCNT and fertilized embryos at the one-cell stage using the cut-off filter of 2.0-fold or greater difference (FIG. 2, 1A+1B). This corresponds to only ˜1.6% of the detected transcripts, indicating that the transcriptome of cloned one-cell embryos is very close to that of controls. Of the 259 differentially expressed mRNAs, 137 were higher in SCNT than in fertilized embryos (FIG. 2, 1A), whereas 122 were lower (FIG. 2, 1B). When considering the transcripts that are different and also sensitive to the α-amanitin treatment, however, the numbers decreased to 45 and 8, respectively. Three mRNAs (Fos, Yy1, Zfp352) were tested by qRT-PCR and all confirmed to be elevated and α-amanitin-sensitive in SCNT embryos, indicating aberrant transcription and mRNA accumulation even at this early stage. As many as 80% of the differentially expressed mRNAs (206 out of 259) were indeed not diminished by α-amanitin treatment, and thus were likely of maternal origin. Three well-known maternal transcripts (Zar1, H1foo and Cpa1) were confirmed by qRT-PCR to be present at a reduced abundance in SCNT embryos when compared to normal embryos (FIG. 1), providing further evidence that these maternal mRNAs are indeed affected. It should be noted that the real time RT-PCR data did not reveal any effect of culture media in this experiment for H1foo or any of these three maternal mRNAs (FIG. 1). These observations indicate that the donor cell genome is markedly silenced by the ooplasm at this point in development, and that regulation of maternal mRNA stability, and possibly translation, is altered in clones with some maternal mRNAs being stabilized and others being precociously degraded.

Relationship Between Genes Affected at the One-Cell Stage and Specific Biological Processes:

Inventors sought to determine whether any specific biological processes were likely affected by the differential effects on the maternal mRNA population. Inventors attempted to divide the list of differentially expressed maternal mRNAs into functional categories. Of the 114 maternal mRNAs that were of lower abundance in clones (FIG. 2, 1E), 59 had some annotation information attached to them. Inventors did not, however, find any specific gene ontology (GO) category that included more than four transcripts in the list.

Out of the 16 mRNAs (FIG. 2, 1H) that were expressed more highly in SCNT embryos as compared to both fertilized and parthenogenetic embryos in an α-amanitin-sensitive manner, ten were annotated. In sharp contrast to the maternal mRNAs, these ten mRNAs displayed a clear bias in functional category, four encoding transcription factors (9030612M13Rik, Dbp, Fos, Gadd45g), and one additional mRNA (Zfp352) encoding a putative transcription factor (Liu et al, 2003). Inventors tested and confirmed the differential expression of two of these transcripts by qRT-PCR (Fos and Zfp352; FIG. 1). Among the six mRNAs that were more highly expressed in fertilized embryos as compared to either SCNT or parthenogenetic embryos in an α-amanitin-sensitive manner (FIG. 2, 1L), none encoded transcription factors.

To determine whether the 16 genes examined in FIG. 2, and over-expressed in SCNT embryos, reflected gene activity of the donor nuclei we examined a microarray data set for cumulus cells. These cumulus cells were isolated from cumulus-oocyte complexes (COCs) obtained from PMSG-primed 22-day-old females. Additional samples corresponded to cells isolated from the COCs of 12 d old females and cultured for 10 d in vitro as described (O'Brien et al., 2003). Of the 16 genes overexpressed in SCNT embryos, 13 were among those detected as being expressed in samples of cells isolated directly from 22 d COCs, and one additional gene was expressed in the in vitro cultured cells. One additional gene (Zfp352) was confirmed qRT-PCR (FIG. 1) to be expressed in cumulus cells from ovulated cumulus-oocyte complexes (donors employed for SCNT). The remaining transcript (C130047D21Rik) was not detected in the Eppig array data, and is not included among available ABI TaqMan gene expression IDs, and so was not tested by qRT-PCR. Thus, of the 16 genes that were transcribed and over-expressed in one-cell SCNT embryos, at least 15 are expressed in cumulus cells. This indicates that the array of genes overexpressed in one-cell SCNT embryos correlates highly with the gene activity of the donor nuclei.

Global Changes in Gene Expression During the Second Embryonic Cell Cycle:

The overall array of different transcripts in both SCNT and fertilized embryos increased at the two-cell stage compared to the one-cell stage. In fertilized embryos, for example, the percent P-call increased from an average of 34.9 to an average of 37.5. Similarly, for SCNT embryos this value increased from 36.9 to 40.1 (Table 2, “% P call”). By contrast, for α-amanitin treated samples, no such increases were seen, and in fact the overall transcriptome complexity diminished during this period. Inventors also observed a much larger difference between the average number of transcripts detected in untreated and α-amanitin treated SCNT embryos than between untreated and α-amanitin treated fertilized embryos (9.9% and 5.7%, respectively), and SCNT embryos exhibited a larger array of transcripts than fertilized embryos (p<0.01). These results reflect activation of the embryonic genome, leading to a net increase in the complexity of the transcript population, and indicate that SCNT embryos transcribe an expanded array of genes at the two-cell stage as compared to fertilized or parthenogenetic controls.

Inventors indeed observed substantial differences between the transcriptomes of SCNT embryos and fertilized embryos (FIG. 3), and this was about an order of magnitude greater than the difference observed at the one-cell stage. We found 2,427 mRNAs differentially expressed between SCNT and normal embryos (FIG. 3, 2A+2B). Of these, ˜67% (1,633) were over-expressed in SCNT embryos (FIG. 3, 2A), and 33% (794) were under-expressed relative to fertilized embryos (FIG. 3, 2B). Of the 1,633 over-expressed mRNAs in SCNT embryos, 1,087 (67%) were α-amanitin-sensitive (FIG. 3, 2D), and hence actively transcribed, whereas 546 (33%) were not diminished by α-amanitin treatment (FIG. 3, 2C). Of the 794 mRNAs that were expressed at reduced abundances in SCNT embryos (FIG. 3, 2E+2F), 452 (57%) were transcribed (FIG. 3, 2F) and 342 (43%) were not diminished by α-amanitin treatment (FIG. 3, 2E).

To determine the degree to which the large differences between SCNT and fertilized embryos was the result of unique properties of SCNT embryos, or instead might be due to differences related to egg activation, absence of a fertilizing sperm, or simple effects of developmental timing, we examined in parthenogenetic embryos expression of mRNAs that were differentially transcribed between SCNT and fertilized embryos. Parthenogenetic embryos were prepared from the same pools of oocytes as SCNT embryos, activated in synchrony, and cultured in parallel, and also lack any fertilizing sperm contribution. The expression of 880 (81%) of 1,087 mRNAs that were transcriptionally elevated in SCNT embryos relative to fertilized embryos was also elevated relative to parthenogenetic controls (FIG. 3, 2J). None of these was media-sensitive. Of the 452 transcribed mRNAs that were reduced in expression in SCNT embryos relative to fertilized embryos, a majority (302, 67%) was likewise reduced in SCNT embryos relative to parthenogenetic embryos (FIG. 3, 2M). Seven of these were among the media-sensitive list of genes. These results indicate that the defects in gene expression detected in SCNT embryos are due to unique features of cloned embryos, and not due to absence of a sperm, or an effect of the egg activation protocol or developmental timing.

In addition to the above effects on transcribed genes, we observed significant differences between clones and both fertilized and parthenogenetic controls in the population of non-transcribed, maternal mRNAs (FIG. 3, 2H and 2L). The vast majority of these differences were insensitive to culture media.

Relationship Between Genes Differentially Transcribed at the Two-Cell Stage and Specific Biological Processes:

The large number of genes differentially expressed between SCNT and control two-cell embryos raises the question as to whether the aberrant regulation of these affected genes alters specific biological processes in SCNT embryos, and hence can account for some of the unusual characteristics observed for SCNT embryos. Inventors analyzed the lists of differentially expressed genes using three different computational approaches. The first approach applied the Expression Analysis Systematic Explorer (EASE) software (Table 3). Among the transcripts over-represented in SCNT embryos, EASE analysis identified 13 Gene Ontology (GO) categories with an EASE score <0.05 (Table 3). Oxidoreductase activity was the category identified with the most significant level of over-representation, and the transporter activity category presented the largest number (68) of affected genes within a category. According to the EASE analysis of the 302 mRNAs that were reduced in expression in SCNT embryos relative to control embryos, there was only one GO category (nucleic acid binding) significantly over-represented (EASE score, 0.00295, n=155 genes).

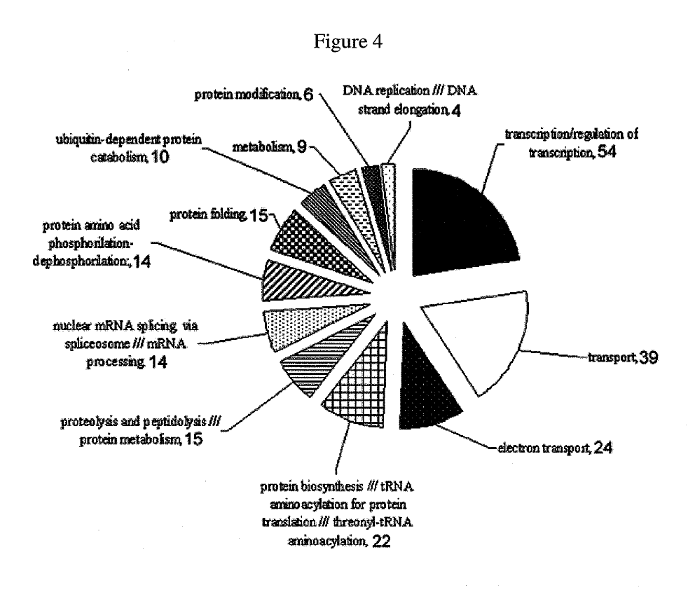

EASE analysis is limited by the degree and accuracy of annotations within category. Moreover it relies solely on numerical relationships between genes lists, it does not account for magnitudes of changes of individual genes, and cannot account for differences in arrays of genes within categories. Hence, although a positive result with EASE analysis provides clear evidence that a specific process is affected, a negative result does not exclude other biologically relevant differences. Inventors evaluated the lists of differentially expressed mRNAs using a second approach to understand what processes may be operating during early embryogenesis and altered by SCNT. The transcripts in each list of differentially expressed mRNAs were assigned to functional categories and then the categories with the higher number of entries analyzed, regardless of their relative overrepresentation (EASE) value (FIG. 4). Inventors also took into account the array of genes within each category.

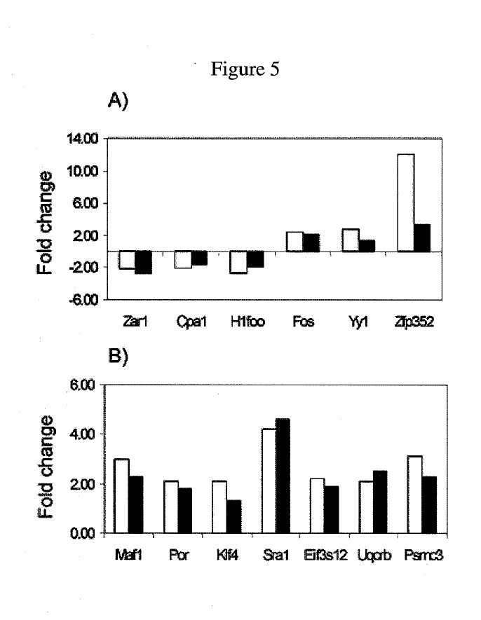

Of the 466 transcripts that have a GO annotation assigned to them, the most abundant category represented was that of transcription factors (TF) and transcriptional regulators (54 transcripts). The 54 TF mRNAs over-expressed in SCNT embryos were elevated by ratios ranging from 2 to 12.7 fold. We tested and confirmed by qRT-PCR analysis the increased expression of Klf4, Maf1 and Sra1 mRNAs (FIGS. 1, 5). The next largest categories encompassed transcripts involved in transport across membranes (39 transcripts) and by transcripts involved in the oxidative phosphorylation pathway (24 transcripts), thus confirming the results of the EASE analysis for these two categories. The qRT-PCR analysis confirmed increased expression of Uqcrb and Por (electron transport), Psmc3 (transport) and Eif3e12 (protein biosynthesis; FIGS. 1, 5). It is noteworthy that the 24 transcribed and overexpressed members of the oxidative phosphorylation category are all encoded by nuclear genes and are distributed among all of the OXPHOS protein complexes. Additional categories up-regulated in SCNT embryos were those of proteolysis, peptidolysis, protein phosphorylation and dephosphorylation, and protein folding.

Among the 302 α-amanitin-sensitive mRNAs that were reduced in expression in SCNT embryos (FIG. 3, 2M) relative to control embryos, 169 were annotated. Interestingly, the transcription factor category was once again the largest category (n=35), indicating further deficiencies in transcription regulation in SCNT embryos. This category was followed by transport across membrane (n=18), and by proteolysis (n=8) and protein biosynthesis (n=7).

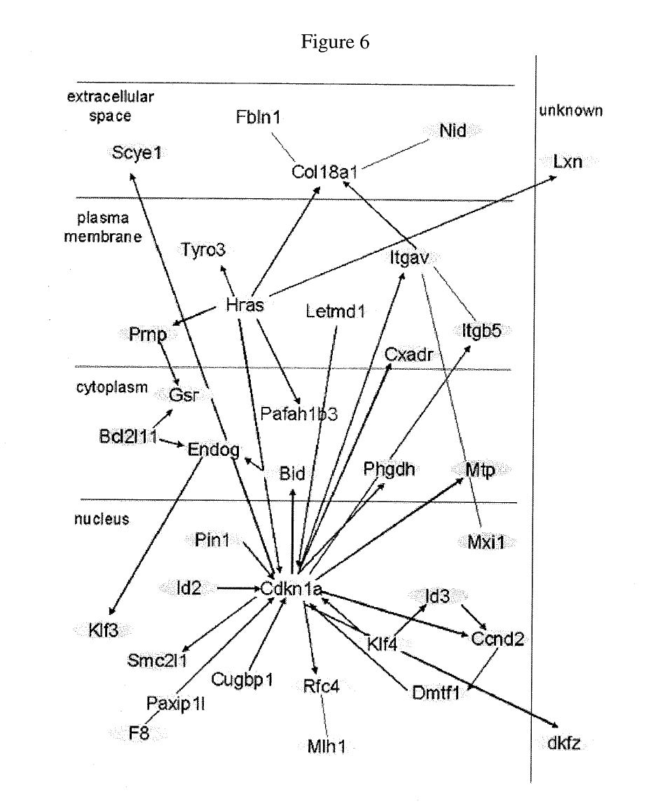

As described above, the TF category was the largest category of affected genes identified by our manual assignment of genes to functional categories. The combinatorial nature of interactions among transcription factors raises the potential that perturbations in TF expression could have a far-reaching effect on the overall process of nuclear reprogramming. We therefore used Ingenuity Pathway Analysis (IPA) to determine networks of genes that may interact with the transcription factors whose expression was perturbed in SCNT embryos. IPA identified 15 networks linking the affected TFs either directly or indirectly to other affected target genes, or indicating direct interaction between different TFs within the affected list. In the list of 54 TFs 42 had scientific literature and annotation available, while 12 lacked information on interaction with other transcripts. Thirty-three of the 42 annotated TFs (79%) were identified by IPA as interacting with other TFs (31, 74%) and/or other genes in the list of upregulated transcripts (16, 38%). A representative example of such networks is presented in FIG. 6.

Discussion:

The data presented here provide for the first time in any species a detailed insight into the extent and timing of nuclear reprogramming during the first two cell cycles of development, reveal substantial disregulation of both transcription and maternal mRNA handling, and identify specific cellular processes that are affected by these defects. With respect to the extent of nuclear reprogramming, our data reveal that, although transcription in the donor nucleus appears to be greatly extinguished by the late one-cell, the donor cell genome nevertheless manifests itself via transcription and accumulation of a small array of transcripts. During the second cell cycle, when the rate of embryonic gene transcription normally increases, the donor cell genome directs the aberrant expression of over 1,000 different transcripts (880 also elevated relative to parthenotes), and deficient transcription of many other genes. These results are consistent with the previously reported dramatic differences in SCNT embryo phenotype as compared to fertilized or parthenogenetic control embryos (Gao et al., 2003, 2004a; Ng and Gurdon, 2005). Inventors previously reported that clones display altered phenotypes even before the first cell division (Chung et al., 2002), and this early effect of the donor cell genome is evident in the microarray data as well.

Superimposed on this deficiency in transcriptional reprogramming is a substantial disruption in the maternal mRNA population, with a large number of maternal mRNAs being either precociously degraded or failing to undergo degradation. Among the transcripts differentially expressed between SCNT and fertilized embryos at the two-cell stage, 888 (37% of the total) were not diminished by α-amanitin treatment, and therefore were likely of maternal origin (FIG. 3, 2C+2E); over 40% (373) of these are also affected relative to parthenotes (FIG. 3, 2H+2L). This effect on the maternal mRNA population appears to be an intrinsic feature of clones, and not an effect of the culture system, because only 5 of these mRNAs was affected at the two-cell stage by choice of culture medium, and one of these (H1foo) was also reduced in one-cell SNCT embryos, but was not media-sensitive at that stage. Of the 373 affected maternal mRNAs, 104 were reduced in SCNT embryos and thus appeared to be precociously degraded. This accelerated degradation at the two-cell stage may be of comparatively little consequence to the embryo, because it may have little effect on expression of proteins that are being eliminated at that stage. For example, the H1foo mRNA encodes a protein that becomes undetectable in embryonic nuclei at the two-cell stage in both controls and SCNT embryos (Gao et al., 2004b). Of much greater potential significance, we observed a large number of maternal mRNAs that were elevated in SCNT embryos (269 mRNAs elevated in clones relative to both normal and parthenogenetic embryos; FIG. 3, 2H). These mRNAs most likely represent maternal transcripts that are inappropriately stabilized in the SCNT embryo. Although it is possible that some of these mRNAs correspond to abundant mRNAs in the donor cell cytoplasm and are transferred along with the nucleus, this is unlikely for several reasons. First, the donor cell is quite small in comparison to the oocyte and much of its cytoplasm is removed before injection. Thus, it is unlikely that mRNAs in the cumulus donor can make a substantial contribution to the array result. Second, we observe that many mRNAs that are expressed in somatic cells (even at high levels) but present at very low abundances in eggs (e.g., actin, Hprt, Pdha1, Pgk1, Prps1, Xist) are not elevated in clones. Third, it is most unlikely that such a large number of affected mRNAs would be abundant enough in cumulus cells to raise the observed abundance in clones. Fourth, we observe that only 92 mRNAs are elevated and α-amanitin-insensitive at the one-cell stage, but 269 are affected at the two-cell stage (FIG. 5, 1C and FIG. 3, 2H), an unlikely pattern if the source was solely the donor cell. Last, in favor of the explanation that these mRNAs are stabilized in clones, we find that 159 (59%) of the 269 α-amanitin-insensitive, affected mRNAs increase in relative abundance between the one-cell and two-cell stage, indicating a greater stability relative to the rest of the maternal mRNA population. Of the remaining mRNAs, 87 (32%) do not change significantly in abundance from the one-cell to the two-cell stage, also indicating long-term stability. Only 23 (9%) decrease in abundance during this period. These observations indicate that the majority of elevated, α-amanitin-insensitive mRNAs in SCNT embryos are very likely maternal in origin rather than imported with the somatic nucleus. Thus, cloned embryos do not undergo the normal elimination of a large number of maternal mRNAs that occurs in fertilized and parthenogenetic control embryos.

The precocious loss or stabilization of a large number of maternal mRNAs in clones was totally unanticipated. Although the molecular basis for this phenomenon is unknown, it is possible that the embryonic genome coordinates maternal mRNA degradation. Consistent with this proposal is that α-amanitin treatment apparently stabilizes some maternal mRNAs (Worrad and Schultz, 1997; Rambathala et al, 1995). Replacing an embryonic genome with a somatic cell genome, with attendant aberrant gene regulation, could therefore lead to such defects. This explanation seems less likely for the one-cell stage, because only a small number of genes are aberrantly transcribed at this stage.

Depletion of factors associated with the spindle-chromosome complex (SCC), which is removed during the first step of cloning, could be a contributing factor. Tetraploid embryos, prepared identically to clones but without SCC removal, display ameliorated effects of the somatic cell genome (Gao et al, 2003), including a lack of aberrantly expressed somatic cell type DNMT1, reduced glucose uptake, reduced requirement for glucose in the culture medium, reduced expression of GLUT4, correct regulation of GLUT1 localization to the plasma membrane, and a much greater tolerance for embryo culture medium (Gao et al, 2003). In addition, the presence or absence of the SCC affects the pace at which the oocyte loses the ability to direct changes in histone H1 composition (Chung et al, 2003). Thus, absence of the regulatory functions of the SCC could contribute to the observed disruption in maternal mRNA stability, particularly at the one-cell stage.

The combined effects of aberrant transcription and mRNA handling disrupt the array of mRNAs that direct a range of specific cellular processes. The largest group of affected transcripts encodes transcription and mRNA processing factors—we observe this at both the 1- and two-cell stages—such that some transcription factor genes normally transcribed in normal embryos are under-expressed in SCNT embryos. The relative abundances of mRNAs that regulate mRNA localization and transport were also reduced in SCNT embryos. Thus, SCNT embryos exhibit profound deficiencies in transcriptional reprogramming. This, coupled with a deficiency in post-transcriptional processes, could readily result in the observed aberrant phenotype of SCNT embryos.

Reprogramming of transcription factors may be a difficult step in cloning because these proteins are responsible for establishing and maintaining a stable differentiated state of the donor somatic cell, and thus must themselves be programmed for stable expression. Genes that define a cell state are often among the most stable with respect to expression programming. In Drosophila for example, genes involved in egg polarity, and gap, pair rule, and segmentation genes act in a sequential manner to establish a combinatorial program of expression of target transcription regulatory genes (e.g., Hox genes), which become programmed for expression in a stable spatial pattern even after the patterning genes cease to be expressed (Gilbert, 2000). This involves the actions of chromatin regulatory genes (e.g., Polycomb) that establish a stable chromatin structure. Thus, cloned embryos may be predisposed to over-express genes encoding transcription factors. This would lead to aberrant expression of numerous other downstream target genes, thus affecting cloned embryo phenotype. Conversely, clones should also exhibit deficiencies in expression of TF genes associated with the embryonic state.

The results presented here support this proposal. For example, inventors observed an entire network of transcriptional regulators and their affected downstream genes to be upregulated in clones. Moreover, several of the aberrantly transcribed transcription factor genes, either in this network or otherwise, fit the profile of genes that establish cell state by regulating a wide array of target genes. Excellent examples of these are Sra1 (NM_{—}025291), Klf4 (NM_{—}010637), and Cbx4 (NM_{—}007625). Sra1 is expressed in all human tissues examined and encodes an RNA component of ribonucleoprotein complexes that contain steroid receptor coactivator-1 and may confer specificity on these transcriptional complexes (Lanz et al, 1999). KLF4 (GKLF) is likewise widely expressed, participates in epithelial cell differentiation (Segre et al, 1999; Jaubert et al, 2003), exerts anti-proliferative, pro-differentiative effects in many cell types (Siddique et al, 2003; Liu et al, 2003a,b; Hiddenbusch et al, 2004; Higaki et al, 2002; Chen et al, 2002a; Chen et al, 2002b; Chen et al, 2003; Foster et al, 2005; Li et al, 2005; Liu et al, 2005; Wu et al, 2004; Katz et al, 2005; Yoon et al, 2005), and regulates a wide variety of genes (Jaubert et al, 2003; Siddique et al, 2003; Liu et al, 2003a,b; Hinnebusch et al, 2004; Higaki et al, 2002; Chen et al, 2003; Zhang et al, 2005; Yasuda et al, 2002; Basu et al, 2004; Liu et al, 2005; Piccinni et al, 2004; Blanchon et al, 2001; Chiambaretta et al, 2004; Chen et al, 2002a; Ai et al, 2004; Miller et al, 2001; Mao et al, 2003; Reidling et al, 2003). KLF proteins also interact with multiple other transcription factors, such as FLH3 and CtBP2 (Turner et al, 2003; van Vliet et al, 2000l; Yang et al, 2005; Schoy et al, 2000; Gallagher et al, 2000; Sabath et al, 1996; Crossley et al, 1996; Turner et al, 1998). We also observed increased expression of the Cbx4 mRNA in our microarray data. The CBX4 protein, like KLF4, affects the expression of a myriad of genes, through its role in the formation of Polycomb bodies, effects on chromatin structure, recruitment of various factors to these complexes, and a combination of either activating or repressive effects (e.g., Kagey et al, 2003; Kagey et al, 2005; Long et al, 2005; Satjin et al, 1997). The ability of both KLF4 and CBX4 to recruit CtBP to regulatory complexes suggests possible cooperative interactions between these proteins.

Another striking category of aberrantly expressed genes included those involved in oxidative phosphorylation. Genes encoding components of all of the OXPHOS protein complexes are up-regulated in clones, with some mRNAs overexpressed as a result of transcription and some elevated as a result of maternal mRNA stabilization. This may exert an effect on carbohydrate metabolism and energy production in clones. Indeed, we have reported previously that clones display increased glucose uptake and a strong preference for glucose-containing media. In this regard, it is interesting to note that one of the genes known to affect mitochondria transcription, Tfam, is present in the list of elevated genes in two-cell SCNT embryos, further supporting the concept of a “ripple effect” of altered reprogramming of transcription factor on downstream genes and embryonic phenotype.

Another prominent affected category encodes proteins related to solute transport and homeostasis. We observe a large number of over-expressed mRNAs at the two-cell stage in this category, and also a large number of maternal mRNAs that are aberrantly stabilized at the two-cell stage. This indicates that the cellular mechanisms regulating ion transport, amino acid transport, intracellular pH, and osmolarity are likely altered. This would likely contribute to the previously reported preference of clones for somatic cell culture media (Chung et al, 2002), which differ a great deal from embryo culture media with respect to ionic and amino acid composition.

With such a large number of aberrantly transcribed genes, the question arises whether so many genes are mis-expressed under the control of a large number or a limited number of transcription regulatory mechanisms. In addition to the possible “ripple effect” that may arise downstream of mis-regulated transcription factor encoding genes, the possibility exists that factors expressed in the oocyte may contribute to aberrant gene regulation. The two-cell stage constitutes a period of transcriptional promiscuity during which very little histone H1 linker of any type exists, and during which the ability to regulate gene transcription is evolving (Wiekowsly et al., 1997). Given the different chromatin structure of somatic cell nuclei as compared to gamete genomes, these conditions establish the possibility that ooplasmic factors may initially activate a range of genes in the somatic nucleus that might not otherwise be activated in the normal embryo. Such activation could have broad-reaching effects, particularly when combined with the downstream consequences of aberrant transcription factor gene expression.

The observations presented here provide vital new information for evaluating the mechanisms and limitations of nuclear reprogramming during somatic cell nuclear transfer. These data also provide a rich foundation for understanding the basic biology of ooplasmic-nuclear interactions, the biology of cloning, and specific factors that must be considered if the process is to be improved.

TABLE 2
Quality control parameter for array hybridization in different kind of
embryos and treatment.

| | | | one- | | | | | two- | | |
|---|---|---|---|---|---|---|---|---|---|---|
| | | | cell | | | | | cell | | |
| Parameter | Fert. | Fert. + a | SCNT | SCNT + a | Parth. | Fert. | Fert. + a | SCNT | SCNT + a | Parth. |
| Scale factor | 1.4-2.9 | 0.6-1 | 1.3-1.9 | 1.7-2.3 | 0.9-1.1 | 0.8-1 | 1.3-1.76 | 0.7-1 | 1.6-2.2 | 1.4-1.7 |
| Background | 35.8-50.5 | 47.6-63.1 | 35.8-40.7 | 37.5-47.4 | 55.1-63.4 | 46.1-64.5 | 51.8-62.5 | 53.2-63.9 | 41-49.8 | 49.5-60.3 |
| % P call | 33.5-36.1 | 38-39 | 35.5-37.6 | 33.2-35.2 | 37.2-38.2 | 36.2-38.8 | 31.4-32.3 | 39.5-41.1 | 29.4-31.4 | 34.5-36 |
| Actin 3/5 | 3.3-12.4 | 3.7-4.4 | 4.8-15 | 4.2-13.9 | 3.9-5.5 | 4.4-7.2 | 4.2-5.9 | 4.9-7.4 | 4.2-4.9 | 4.7-5.2 |
| GAPDH 3/5 | 1.5-6.2 | 5.6-6.2 | 1.7-6.2 | 1.9-7.7 | 5.1-6.2 | 5.6-7.2 | 4.2-6.1 | 4.2-6.8 | 4-6.2 | 4.7-6.6 |

a = α-amanitin in culture medium;
Fert. = fertilized embryos;
SCNT = somatic cell nuclear transfer embryos;
Parth. = parthenogenetic embryos.

TABLE 3
EASE analysis output for genes upregulated at the two-cell stage in SCNT
embryos and sensitive to α-amanitin treatment (FIG. 3, Set 2J).
Characteristic molecular functions are listed for annotated genes with an
EASE score <0.05.

| GO Molecular Function | EASE score | N. genes |
|---|---|---|
| oxidoreductase activity | 1.20E−05 | 50 |
| electron transporter activity | 1.28E−04 | 18 |
| NADH dehydrogenase activity | 6.96E−05 | 9 |
| oxidoreductase activity\, acting on NADH or | 1.57E−04 | 11 |
| NADPH | | |
| transporter activity | 2.53E−03 | 68 |
| primary active transporter activity | 2.05E−03 | 19 |
| carrier activity | 5.37E−04 | 32 |
| ion transporter activity | 2.49E−03 | 33 |
| cation transporter activity | 2.52E−03 | 30 |
| monovalent inorganic cation transporter activity | 5.75E−05 | 20 |
| sodium ion transporter activity | 2.28E−04 | 8 |
| hydrogen ion transporter activity | 1.34E−04 | 19 |

### REFERENCES

Aghayan, M., Rao, L. V., Smith, R. M., Jarett, L., Charron, M. J., Thorens, B., Heyner, S., 1992. Developmental expression and cellular localization of glucose transporter molecules during mouse preimplantation development. Development 115:305-12
Ai, W., Liu, Y., Langlois, M., Wang, T. C., 2004. Kruppel-like factor 4 (KLF4) represses histidine decarboxylase gene expression through an upstream Sp1 site and downstream gastrin responsive elements. J Biol. Chem. 279: 8684-93.
Al-Sarraj, A., Day, R. M., Thiel, G. 2005. Specificity of transcriptional regulation by the zinc finger transcription factors Sp1, Sp3, and Egr-1. J Cell Biochem. 94:153-67.
Baltz, J. M., Biggers, J. D., Lechene, C., 1991a. Relief from alkaline load in two-cell stage mouse embryos by bicarbonate/chloride exchange. J Biol Chem 266:17212-7
Baltz, J. M., Biggers, J. D., Lechene C., 1991b. Two-cell stage mouse embryos appear to lack mechanisms for alleviating intracellular acid loads. J Biol Chem 266:6052-7
Baltz, J. M., Biggers, J. D., Lechene, C., 1993. A novel H+ permeability dominating intracellular pH in the early mouse embryo. Development 118:1353-61
Basu, P., Sargent, T. G., Redmond, L. C., Aisenberg, J. C., Kransdorf, E. P., Wang, S. Z., Ginder, G. D., Lloyd, J. A., 2004. Evolutionary conservation of KLF transcription factors and functional conservation of human gama-globin gene regulation in chicken. Genomics 84:311-9.
Blanchon, L., Bocco, J. L., Gallot, D., Gachon, A. M., Lernery, D., Dechelotte, P., Dastugue, B., Sapin, V., 2001. Co-localization of KLF6 and KLF4 with pregnancy-specific glycoproteins during human placenta development. Mech Dev 105:185-9.
Campbell, K. H., Alberio, R., Choi, I., Fisher, P., Kelly, R. D., Lee, J. H., Maalouf, W., 2005. Cloning: eight years after Dolly. Reprod. Dometic. Anim. 40: 256-68.
Carayannopoulos, M., Phay, J., Cui, Y., Moley, J., Moley, M., 2001. Cloning and molecular characterization of mouse GLUT9, a new embryonically expressed glucose transporter. SSR meeting abstracts.
Carayannopoulos, M. O., Chi, M. M., Cui, Y., Pingsterhaus, J. M., McKnight, R. A., Mueckler, M., Devaskar, S. U., Moley, K. H., 2000. GLUT8 is a glucose transporter responsible for insulin-stimulated glucose uptake in the blastocyst. PNAS 97:7313-8
Chen, Z. Y., Shie, J. L., Tseng, C. C., 2002a. STAT1 is required for IFN-gamma-mediated gut-enriched Kruppel-like factor expression. Exp Cell Res. 281:19-27.
Chen, Z. Y., Shie, J. L., Tseng, C. C., 2002b. Gut-enriched Kruppel-like factor represses ornithine decarboxylase gene expression and functions as checkpoint regulator in colonic cancer cells. J Biol. Chem. 277: 46831-9.
Chen, X., Whitney, E. M., Gao, S. Y., Yang, V. W., 2003. Transcriptional profiling of Kruppel-like factor 4 reveals a function in cell cycle regulation and epithelial differentiation. J Mol Biol 326: 665-77.
Chi, M. M., Pingsterhaus, J., Carayannopoulos, M., Moley, K. H., 2000. Decreased glucose transporter expression triggers BAX-dependent apoptosis in the murine blastocyst. J Biol Chem 275:40252-7
Chiambaretta, F., De Graeve, F., Turet, G., Marceau, G., Gain, P., Dastugue, B., Rigal, D., Sapin, V., 2004. Cell and tissue specific expression of human Kruppel-like transcription factors in human ocular surface. Mol V is 10: 901-9.
Chung, Y. G., Mann, M. R., Bartolomei M S, Latham K E. 2002. Nuclear-cytoplasmic “tug of war” during cloning: effects of somatic cell nuclei on culture medium preferences of preimplantation cloned mouse embryos. Biol Reprod. 66:1178-84.
Chung, Y. G., Ratnam, S., Chaillet, J. R., Latham, K. E. 2003. Abnormal regulation of DNA methyltransferase expression in cloned mouse embryos. Biol Reprod. 69:146-53.
Crossley, M., Whitelaw, E., Perkins, A., Williams, G., Fujiwara, Y., Orkin, S. H., 1996. Isolation and characterization of the cDNA encoding BKLF/TEF-2, a major CACCC-box-binding protein in erythroid cells and selected other cells. Mol Cell Biol 16: 1695-705.
Edwards, L. J., Williams, D. A., Gardner, D. K., 1998. Intracellular pH of the mouse preimplantation embryo: amino acids act as buffers of intracellular pH. Hum Reprod 13:3441-8
Edwards, L. J., Williams, D. A., Gardner, D. K., 1998. Intracellular pH of the preimplantation mouse embryo: effects of extracellular pH and weak acids. Mol Reprod Dev 50:434-42.
Foster, K. W., Liu, Z., Nail, C. D., Li, X., Fitzgerald, T. J., Bailey, S. K., Frost, A. R., Louro, I. D., Townes, T. M., Paterson, A. J., Kudlow, J. E., Lobo-Ruppert, S. M., Ruppert, J. M., 2005. Induction of KLF4 in basal keratinocytes blocks the proliferation-differentiation switch and initiates squamous epithelial dysplasia. Oncogene 24: 1491-500.
Gallagher, P. G., Romana, M., Tse, W. T., Lux, S. E., Forget, B. G., 2000. The human ankyrin-1 gene is selectively transcribed in erythroid cell lines despite the presence of a housekeeping-like promoter. Blood. 96:1136-43.
Gao, S., Latham, K. E. 2004. Maternal and environmental factors in early cloned embryo development. Cytogenet Genome Res. 105:279-84.
Gao, S., Chung, Y. G., Williams, J. W., Riley, J., Moley, K., Latham, K. E. 2003. Somatic cell-like features of cloned mouse embryos prepared with cultured myoblast nuclei. Biol Reprod. 69:48-56.
Gao, S., Czirr, E., Chung, Y. G., Han, Z., Latham, K. E. 2004a. Genetic variation in oocyte phenotype revealed through parthenogenesis and cloning: correlation with differences in pronuclear epigenetic modification. Biol Reprod. 70:1162-70.
Gao, S., Han, Z., Kihara, M., Adashi, E., Latham, K. E. 2005. Protease inhibitor MG132 in cloning: no end to the nightmare. Trends Biotechnol. 23:66-8.
Gao, S., Chung, Y. G., Parseghian, M. H., King, G. J., Adashi, E. Y., Latham, K. E. 2004b. Rapid H1 linker histone transitions following fertilization or somatic cell nuclear transfer: evidence for a uniform developmental program in mice. Dev Biol. 266:62-75.
Gilbert, S. F 2000. Developmental Biology. Sinauer Associates Publishers, Sunderland, Mass. 287-89.
Hagemann, C., Meyer, C., Stojic, J., Eicker, S., Gerngras, S., Kuhnel, S., Roosen, K., Vince, G. H. 2006. High efficiency transfection of glioma cell lines and primary cells for overexpression and RNAi experiments. J Neurosci Methods. [Epub ahead of print].
Higaki, Y., Schullery, D., Kawata, Y., Shnyreva, M., Abrass, C., Bomsztyk, K., 2002. Synergistic activation of the rat laminin gammal chain promoter by the gut-enriched Kruppel-like factor (GKLF/KLF4) and Sp1. Nucleic Acids Res 30: 2270-9.
Hillman, N., Tasca, R. J., 1969. Ultrastructural and autoradiographic studies of mouse cleavage stages. Am J Anat 126: 151-73
Hinnebusch, B. F., Siddique, A., Henderson, J. W., Malo, M. S., Zhang, W., Athaide, C. P., Abedrapo, M. A., Chen, X., Yang, V. W., Hodin, R. A., 2004. Enterocyte differentiation marker intestinal alkaline phosphatase is a target gene of the gut-enriched Kruppel-like factor. Am J Physiol Gastrointest Liver Physiol 286: G23-30.
Hogan, B., Beddington, R., Constantini, F., and Lacy, E. 1994. Manipulating the Mouse Embryo: A Laboratory Manual, 2nd Edition. Cold Spring Harbor Press. Pp. 173-178.
Hogan, A., Heyner, S., Charron, M. J., Copeland, N. G., Gilbert, D. J., Jenkins, N. A., Thorens, B., Schultz, G. A., 1991. Glucose transporter gene expression in early mouse embryos. Development 113:363-72.
Hosack, D. A., Dennis, G. Jr., Sherman, B. T., Lane, H. C., Lempicki, R. A. 2003. Identifying biological themes within lists of genes with EASE. Genome Biol 4: 70.
Humpherys, D., Eggan, K., Akutsu, H., Hochedlinger, K., Rideout, W. rd, Biniszkiewicz, D., Yanagimachi, R., Jaenisch, R. 2001. Epigenetic instability in ES cells and cloned mice. Science. 293:95-7.
Humpherys, D., Eggan, K., Akutsu, H., Friedman, A., Hochedlinger, K., Yanagimachi, R., Lander, E. S., Golub, T. R., Jaenish, R. 2002. Abnormal gene expression in cloned mice derived from embryonic stem and cumulus cell nuclei. PNAS 99: 12889-94.
Jaubert, J., Cheng, J., Segre, J. A., 2003. Ectopic expression of kruppel like factor 4 (Klf4) accelerates formation of the epidermal permeability barrier. Development 130: 2767-77.
Kagey, M. H., Melhuish, T. A., Powers, S. E., Wotton, D., 2005. Multiple activities contribute to Pc2 E3 function. EMBO J. 24:108-19.
Kagey, M. H., Melhuish, T. A., Wotton, D., 2003. The polycomb protein Pc2 is a SUMO E3. Cell. 113:127-37.
Katz, J. P., Perreault, N., Goldstein, B. G., Actman, L., McNally, S. R., Silberg, D. G., Furth, E. E., Kaestner, K. H., 2005. Loss of Klf4 in mice causes altered proliferation and differentiation and precancerous changes in the adult stomach. Gastroenterology 128: 935-45.
King, T. J., Briggs, R. 1955. Changes in the nuclei of differentiating gastrula cells, as demonstrated by nuclear transplantation. PNAS 41:321-25.
Lanz, R. B., McKenna, N. J., Onate, S. A., Albrecht, U., Wong, J., Tsai, S. Y., Tsai, M-J., O'Malley, B. W., 1999. A steroid receptor coactivator, SRA, functions as an RNA and is present in an SRC-1 complex. Cell 97:17-27.
Latham, K. E. 2004. Cloning: questions answered and unsolved. Differentiation. 72:11-22.
Latham, K. E. 2005. Early and delayed aspects of nuclear reprogramming during cloning. Biol Cell. 97:119-32.
Latham, K. E., Garrels, J. I., Chang, C. and Solter, D., 1991. Quantitative analysis of protein synthesis in mouse embryos I. Extensive re-programming at the one- and two-cell stages. Development. 112, 921-932.
Latham, K. E., Solter, D., Schultz, R. M., 1992. Acquisition of a transcriptionally permissive state during the one-cell stage of mouse embryogenesis. Dev Biol 149: 457-62.
Leppens-Luisier, G., Urner, F., Sakkas, D., 2001. Facilitated glucose transporters play a crucial role throughout mouse preimplantation embryo development. Hum Reprod 16:1229-36
Li, Y., McClintick, J., Zhong, L., Edenberg, H. J., Yoder, M. C., Chan, R. J., 2005. Murine embryonic stem cell differentiation is promoted by SOCS-3 and inhibited by the zinc finger transcription factor Klf4. Blood 105: 635-7.
Liu, T. Y., Chen, H. H., Lee, K. H., Choo, K. B., 2003a. Display of different modes of transcription by the promoters of an early embryonic gene, zfp352, in preimplantation embryos and in somatic cells. Mol Reprod Dev 64: 52-60.
Liu, Y., Sinha, S., Owens, G., 2003b. A transforming growth factor-beta control element required for SM alpha-actin expression in vivo also partially mediates GKLF-dependent transcriptional repression. J Biol Chem 278: 48004-11.
Liu, Y., Sinha, S., McDonald, O. G., Shang, Y., Hoofnagle, M. H., Owens, G. K., 2005. Kruppel-like factor 4 abrogates myocardin-induced activation of smooth muscle gene expression. J Biol Chem 280: 9719-27.
Long, J., Zuo, D., Park, M., 2005. Pc2-mediated sumoylation of Smad-interacting protein 1 attenuates transcriptional repression of E-cadherin. J Biol Chem 280: 35477-89.
Mann, M. R., Chung, Y. G., Nolen, L. D., Verona, R. I., Latham, K. E., Bartolomei, M. S. 2003. Disruption of imprinted gene methylation and expression in cloned preimplantation stage mouse embryos. Biol Reprod. 69:902-14
Mao, Z., Song, S., Zhu, Y., Yi, X., Zhang, H., Shang, Y., Tong, T., 2003. Transcriptional regulation of A33 antigen expression by gut-enriched Kruppel-like factor. Oncogene. 22:4434-43.
Matsumoto, H., Shoji, N., Sugawara, S., Umezu, M., Sato, E., 1998. Microscopic analysis of enzyme activity, mitochondrial distribution and hydrogen peroxide in two-cell rat embryos. J Reprod Fertil 113: 231-8
Miller, K. A., Eklund, E. A., Peddinghaus, M. L., Cao, Z., Fernandes, N., Turk, P. W., Thimmapaya, B., Weitzman, S. A., 2001. Kruppel-like factor 4 regulates laminin alpha 3A expression in mammary epithelial cells. J Biol. Chem. 276: 42863-8.
Moley, K. H., Chi, M. M., Mueckler, M. M., 1998. Maternal hyperglycemia alters glucose transport and utilization in mouse preimplantation embryos. Am J Physiol 275:E38-47
Morita, Y., Tsutsumi, O., Oka, Y., Taketani, Y., 1994. Glucose transporter GLUT1 mRNA expression in the ontogeny of glucose incorporation in mouse preimplantation embryos. Biochem Biophys Res Commun 199:1525-31
Ng, R. K., Gurdon, J. B., 2005. Epigenetic memory of active gene transcription is inherited through somatic cell nuclear transfer. PNAS 102: 1957-62.
O'Brien, M. J., Pendola, J. K., Eppig, J. J. 2003. A revised protocol for in vitro development of mouse oocytes from primordial follicles dramatically improves their developmental competence. Biol. Reprod. 68, 1682-1686.
Ohgane, J., Wakayama, T., Kogo, Y., Senda, S., Hattori, N., Tanaka, S., Yanagimachi, R., Shiota, K. 2001. DNA methylation variation in cloned mice. Genesis. 30:45-50.
Pan, H., O'brien, M. J., Wigglesworth, K., Eppig, J. J., Schultz, R. M., 2005. Transcript profiling during mouse oocytes development and the effect of gonadotropin priming and development in vitro. Dev Biol 286: 493-506.
Pantaleon, M., Kaye, P. L., 1998. Glucose transporters in preimplantation development. Rev Reprod 3:77-81
Pantaleon, M., Ryan, J. P., Gil, M., Kaye, P. L., 2001. An unusual subcellular localization of GLUT1 and link with metabolism in oocytes and preimplantation mouse embryos. Biol Reprod 64:1247-54
Piccinni, S. A., Bolcato-Bellemin, A. L., Klein, A., Yang, V. W., Kedinger, M., Simon-Assmann, P., Lefebvre, O., 2004. Kruppel-like factors regulate the Lamal gene encoding the laminin alpha1 chain. J Biol Chem 279: 9103-14.
Pore, N., Liu, S., Shu, H. K., Li, B., Haas-Kogan, D., Stokoe, D., Milanini-Mongiat, J., Pages, G., O'Rourke, D. M., Bernhard, E., Maity, A. 2004. Sp1 is involved in Akt-mediated induction of VEGF expression through an HIF-1-independent mechanism. Mol Biol Cell. 15(11):4841-53.
Rambhatla, L., Patel, B., Dhanasekeran, N., Latham, K. E. 1995. Analysis of G protein alpha subunit mRNA abundance in preimplantation mouse embryos using a rapid, quantitative RT-PCR approach. Mol. Reprod. Dev. 41: 314-24.
Reidling, J. C., Said, H. M., 2003. In vitro and in vivo characterization of the minimal promoter region of the human thiamin transporter SLC19A2. Am J Physiol Cell Physiol 285: 633-41.
Rhind, S. M., Taylor, J. E., De Sousa, P. A., King, T. J., McGarry, M., Wilmut, I. 2003. Human cloning: can it be made safe? Nat. Rev. Genet. 4, 855-864.
Ryu S, Zhou S, Ladurner A G, Tjian R. 1999. The transcriptional cofactor complex CRSP is required for activity of the enhancer-binding protein Sp1. Nature. 1999 Feb. 4; 397(6718):446-50.
Sabath, D. E., Koehler, K. M., Yang, W. Q., 1996. Structure and function of the zeta-globin upstream regulatory element Nucleic Acids Res. 24: 4978-86.
Saeed, A. I., Sharov, V., White, J., Li, J., Liang, W., Bhagabati, N., Braisted, J., Klapa, M., Currier, T., Thiagarajan, M., Sturn, A., Snuffin, M., Rezantsev, A., Popov, D., Ryltsov, A.,
Satijn, D. P., Olson, D. J., van der Vlag, J., Hamer, K. M., Lambrechts, C., Masselink, H., Gunster, M. J., Sewalt, R. G., van Driel, R., Otte, A. P., 1997. Interference with the expression of a novel human polycomb protein, hPc2, results in cellular transformation and apoptosis. Mol Cell Biol 17:6076-86.
Scohy, S., Gabant, P., Szpirer, C., Szpirer, J., 2000. Identification of an enhancer and an alternative promoter in the first intron of the alpha-fetoprotein gene. Nucleic Acids Res. 28: 3743-51.
Segre, J. A., Bauer, C., Fuchs, E., 1999. Klf4 is a transcription factor required for establishing the barrier function of the skin. Nat. Genet. 22: 356-360.
Sathananthan, A. H., Trounson, A. O., 2000. Mitochondrial morphology during preimplantational human embryogenesis. Hum Reprod 15:148-59
Shepard, T. H., Muffley, L. A., Smith, L. T., 1998. Ultrastructural study of mitochondria and their cristae in embryonic rats and primate (N. nemistrina). Anat Rec 252: 383-92
Shepard, T. H., Muffley, L. A., Smith, L. T., 2000. Mitochondrial ultrastructure in embryos after implantation. Hum Reprod 15:218-28.
Siddique, A., Malo, M. S., Ocuin, L. M., Hinnebusch, B. F., Abedrapo, M. A., Henderson, J. W., Zhang, W., Mozumder, M., Yang, V. W., Hodin, R. A., 2003. Convergence of the thyroid hormone and gut-enriched Kruppel-like factor pathways in the context of enterocyte differentiation. J Gastrointest Surg 7: 1053-61
Smith, S. L., Everts, R. E., Tian, X. C., Du, F., Sung, L. Y., Rodriguez-Zas, S. L., Jeong, B. S., Renard, J. P., Lewin, H. A., Yang, X., 2005. Global gene expression profiles reveal significant nuclear reprogramming by the blastocyst stage after cloning. PNAS 102: 17582-7.
Turner, J., Crossley, M., 1998. Cloning and characterization of mCtBP2, a co-repressor that associates with basic Kruppel-like factor and other mammalian transcriptional regulators. EMBO J. 17: 5129-40.
Turner, J., Nicholas, H., Bishop, D., Matthews, J. M., Crossley, M., 2003. The LIM protein FHL3 binds basic Kruppel-like factor/Kruppel-like factor 3 and its co-repressor C-terminal-binding protein 2. J Biol Chem 278: 12786127-95.
Tusher, V. G., Tibshirani, R., Chu, G., 2001. Significance analysis of microarrays applied to the ionizing radiation response. PNAS 98: 5116-5121.
Van Winkle, L. J. 2001. Amino acid transport regulation and early embryo development. Biol. Reprod. 64:1-12.
van Vliet, J., Turner, J., Crossley, M., 2000. Human Kruppel-like factor 8: a CACCC-box binding protein that associates with CtBP and represses transcription. Nucleic Acids Res. 2000 May 1; 28(9):1955-62.
Worrad, D. M., Ram, P. T., Schultz, R. M. Regulation of gene expression in the mouse oocyte and early preimplantation embryo: developmental changes in Sp1 and TATA box-binding protein, TBP. Development. 1994 August; 120(8):2347-57.
Wang, Q., and Latham, K. E. 2000. Translation of maternal mRNAs encoding transcription factors during genome activation in early mouse embryos. Biol. Repro. 62: 969-978.
Wiekowski, M., Miranda, M., Nothias, J. Y., DePamphilis, M. L., 1997. Changes in histone synthesis and modification at the beginning of mouse development correlate with the establishment of chromatin mediated repression of transcription. J. Cell Science 110: 1147-58.
Worrad, D. M., Schultz, R. M., 1997. Regulation of gene expression in the premplantation mouse embryo: temporal and spatial pattern of expression of the transcription factor Sp1. Mol Reprod Dev 46: 268-77.
Wu, J., Lingrel, J. B., 2004. KLF2 inhibits Jurkat T leukemia cell growth via upregulation of cyclin-dependent kinase inhibitor p21WAF1/CIP1. Oncogene 2004 Oct. 21; 23(49):8088-96.
Yang, X. O., Doty, R. T., Hicks, J. S., Willerford, D. M., 2003. Regulation of T-cell receptor D beta 1 promoter by KLF5 through reiterated GC-rich motifs. Blood 101: 4492-9.
Yasuda, K., Hirayoshi, K., Hirata, H., Kubota, H., Hosokawa, N., Nagata, K., 2002. The Kruppel-like factor Zf9 and proteins in the Sp1 family regulate the expression of HSP47, a collagen-specific molecular chaperone. J Biol Chem 277:44613-22.
Yin, J., Ma, Z., Selliah, N., Shivers, D. K., Cron, R. Q., Finkel, T. H. 2006. Effective gene suppression using small interfering RNA in hard-to-transfect human T cells. J Immunol Methods. [Epub ahead of print]
Yoon, H. S., Ghaleb, A. M., Nandan, M. O., Hisamuddin, I. M., Dalton, W. B., Yang, V. W., 2005. Kruppel-like factor 4 prevents centrosome amplification following gamma-irradiation-induced DNA damage. Oncogene 24: 4017-25.
Zeng, F., Baldwin, D. A., Schultz, R. M., 2004. Transcript profiling during preimplantation mouse development. Dev Biol 272: 483-96.
Zeng, F., Schultz, R. M., 2005. RNA transcript profiling during zygotic gene activation in the preimplantation mouse embryo. Dev Biol 283: 464-57.
Zhang, P., Basu, P., Redmond, L. C., Morris, P. E., Rupon, J. W., Ginder, G. D., Lloyd, J. A., 2005. A functional screen for Kruppel-like factors that regulate the human gamma-globin gene through the CACCC promoter element. Blood Cells Mol. Dis. 35: 227-35.
Zhao, Y., Baltz, J. M., 1996. Bicarbonate/chloride exchange and intracellular pH throughout preimplantation mouse embryo development. Am J Physiol 271:C1512-20
Zhao, Y., Chauvet, P. J., Alper, S. L., Baltz, J. M., 1995. Expression and function of bicarbonate/chloride exchangers in the preimplantation mouse embryo. J Biol Chem 270:24428-34

What is claimed is:

1. A method of increasing cloning efficiency of embryos, the method comprising manipulating Sp1 target genes expression to obtain a statistically significant increase in a fraction of embryos developing to birth.

2. The method of claim 1, wherein manipulating Sp1 target genes expression is conducted in a donor cell nucleus before nuclear transfer.

3. The method of claim 2, wherein the donor cell is treated with siRNA to Sp1 or CRSP protein subunit mRNAs to suppress the expression of Sp1 or CRSP protein subunits, and thus Sp1 target genes.

4. The method of claim 3, wherein the donor cell is treated with siRNA from about 12 h to about 14 h prior to nuclear transfer.

5. The method of claim 2, wherein a nucleus is isolated from a treated donor cell and introduced into an egg.

6. The method of claim 1, wherein manipulating Sp1 target genes expression is conducted in an egg after nuclear transfer.

7. The method of claim 6, wherein Sp1 protein which is present in the egg is temporary neutralized by treating the egg with a sufficient amount of an Sp1 antibody or a dominant negative form of Sp1 such that aberrant overexpression of Sp1 target genes is reduced while permitting expression of other essential Sp1 target genes.

8. The method of claim 7, wherein Sp1 protein is neutralized during the first 1 to 2 cell cycles in clones followed by restoring Sp1 activity via embryonic Sp1 expression.

9. A kit for increasing cloning efficiency of embryos, the kit comprising at least one of (a) a donor cell treated with siRNA to Sp1 mRNA or (b) an egg which is temporary neutralized by treating the egg with a sufficient amount of an Sp1 antibody or a dominant negative form of Sp1 such that aberrant overexpression of Sp1 target genes is reduced.
